(19) United States
(12) Patent Application Publication
(10) Pub. No.: US 2026/0183028 A1
(43) Pub. Date: Jul. 2, 2026

(54) SLIDING FLEXURES FOR PECTUS EXCAVATUM REPAIR

(71) Applicants: Brigham Young University, Provo, UT (US); Children's Hospital Medical Center, Cincinnati, OH (US)

(72) Inventors: Brandon Sargent, Provo, UT (US); Clark Roubicek, Provo, UT (US); Larry Howell, Orem, UT (US); Victor Garcia, Cincinnati, OH (US)

(21) Appl. No.: 19/438,941

(22) Filed: Jan. 2, 2026

Related U.S. Application Data
(60) Provisional application No. 63/741,353, filed on Jan. 2, 2025.

(51) Int. Cl. A61B 17/80

(52) U.S. Cl. CPC A61B 17/8076; A61B 17/8009

(57) ABSTRACT

An implantable medical device, illustratively an arcuate bar with sliding flexures, for the gradual correction of pectus excavatum in patients.

### CROSS-REFERENCE TO RELATED APPLICATION

The present application claims priority to U.S. Provisional Patent Application Ser. No. 63/741,353, filed Jan. 2, 2025, the disclosure of which is expressly incorporated herein by reference.

### BACKGROUND AND SUMMARY OF THE DISCLOSURE

The present application relates to an implantable medical device including sliding flexures and, more particularly to a bar with sliding flexures for use in pectus excavatum repair.

Compliant mechanisms have been favored over traditional rigid-link mechanisms in some engineering applications due to their low part count, stored strain energy, and simplicity/reliability. Such mechanisms offer potential for increased performance in medical applications, where patient safety is a priority, and where accessing a device after initial placement may be difficult, dangerous, and/or painful. Because of their stored strain energy, compliant mechanisms have the potential to perform their intended function in vivo, with little to no additional input from health professionals, greatly reducing risk and increasing patient satisfaction. They can be used in situations where self-correction (correction without any outside adjustment once the device is inserted) is desired or necessary. These benefits make compliant mechanisms an attractive alternative to correct a pectus excavatum deformity.

A phenomenon often called de-stiffening the chest wall has been observed in patients who have pectus carinatum (PC), or pigeon chest. PC is characterized by a deformed sternum that pushes outward, away from the internal organs in the chest cavity. PC is typically corrected through the use of external, wearable bracing devices. Before the correction process for PC begins, the chest wall has an in initial stiffness that opposes any displacement (like a coil spring). The phenomenon is observed once a constant force is applied to the chest wall by the brace; gradually and over time, the stiffness of the chest wall decreases, and the initial constant force then produces greater displacement. This phenomenon resembles stress relaxation in engineering materials.

The device of the present disclosure suggests that the chest wall de-stiffening phenomenon observed in PC patients will hold true for pectus excavatum patients. Pectus excavatum (PE), or funnel chest, is a deformity of the chest wall, characterized by a deformed sternum that typically produces a fist-sized depression in the chest cavity. It is the most common chest wall deformity, with a rate of 1:300/400 births. This condition can result in exercise intolerance, shortness of breath, and chest pain. It may also result in labored breathing during exercise and overall loss of stamina. The current practice to correct this deformity is called the minimally-invasive Nuss procedure (NP), in which a surgeon takes a stiff metal bar and weaves it through the patient's rib cage and underneath the sternum. The bar is bent to match the shape of the patient's rib cage and rotated into its final position inside the chest cavity. This procedure typically produces instant correction of the deformed sternum, which correction distance far surpasses the region of non-painful skeletal deformation. The bar is placed between the ribs in such a way that one rib on either side of the sternum provides a vertical support to the bar, and the reaction load from the displaced sternum is effectively shifted to the ribs. The ends of the bar are often sutured to the outer ribs in efforts to create a fixed connection between the bar and the patient's body, which helps prevent flipping and jostling of the bar.

Because the NP procedure typically produces complete and immediate correction, in many cases, it also causes extreme pain. Healthcare providers traditionally counteract this side-effect by prescribing opiates; because of the increased chance for opiate addiction, an alternative solution is desired. Additionally, some patients may entirely forgo the operation because of the anticipated pain following the operation.

The illustrative device of the present disclosure, or a bar with sliding flexures, is comparable in size to the Nuss bar and is configured to be inserted into the patient's body using a similar procedure. The illustrative bar with sliding flexures reduces patient pain by extending the PE correction stage to a longer period through a more gradual correction. In addition, the bar of the present disclosure may be applied to different pectus excavatum morphologies. By non-limiting example, the bar of the present disclosure may be used for cases where the morphology presented is asymmetrical. Lastly, the bar with sliding flexures of the present disclosure is more flexible than that of the bar used in the NP. This provides easier implantation, leading to a less dangerous surgical procedure.

According to an illustrative embodiment of the present disclosure, a sliding flexure bar for pectus excavatum repair includes a first end portion extending between a proximal end and a distal end, the first end portion including a channel, a second end portion in spaced relation to the first end portion, the second end portion extending between a proximal end and a distal end, and including a channel, a first flexible joint extending from the distal end of the first end portion, a second flexible joint extending from the distal end of the second end portion, and a center portion connecting to the first flexible joint at a first end and the second flexible joint at a second end, the center portion including a channel. The flexible joints permit the center portion to be moveable between a undeflected state and a deflected state, the center portion extends outwardly in a convex manner from the first and second flexible joint in the undeflected state, and the center portion extends inwardly in a concave manner from the first and second flexible joint in the deflected state.

Further, according to an illustrative embodiment of the present disclosure, a sliding flexure bar for pectus excavatum repair includes a first flexure received in the channel of the center portion on a first end and the channel of the first end portion on a second end, the first flexure having variable stiffness along its length. The sliding flexure bar further includes a second flexure received in the channel of the center portion on a first end and the channel of the second end portion on a second end, the second flexure having variable stiffness along its length. The first flexure the second flexure are slidably actuated from a zone of low stiffness to a zone of higher stiffness, stiffening the first and second flexible joint to move the bar into an undeflected state.

According to another illustrative embodiment of the present disclosure, a system for pectus excavatum repair includes a sliding flexure bar. The sliding flexure bar includes a first end portion extending between a proximal end and a distal end and a second end portion in spaced relation to the first end portion, the second end portion extending between a proximal end and a distal end. The sliding flexure bar further includes a first flexible joint extending from the distal end of the first end portion, a second flexible joint extending from the distal end of the second end portion, and a center portion connecting to the first flexible joint at a first end and the second flexible joint at a second end. The flexible joints permit the center portion to be moveable from a undeflected state to a deflected state, and vice versa. The center portion extends outwardly in a convex manner from the first and second flexible joint, and the center portion extends inwardly in a concave manner from the first and second flexible joint in the deflected state. The sliding flexure bar further includes a first flexure including a first end received in the center portion, and a second end received in the channel of the first end portion, the first flexure having variable stiffness along its length, and a second flexure including a first end received in the center portion, and a second end received in the second end portion, the second flexure having variable stiffness along its length.

The illustrative system for pectus excavatum repair further includes a pulling device. The pulling device may be placed externally of the sliding flexure bar and upon actuation increase the force that the center portion exerts to move from the deflected state to the undeflected state. Additionally, increasing the force that the center portion exerts assists actuating the first and second flexures, moving the center portion from a deflected state to an undeflected state.

According to a further illustrative embodiment of the present disclosure, a method of correcting pectus excavatum includes the step of providing a sliding flexure bar. The sliding flexure bar includes a first end portion, a second end portion in spaced relation to the first end portion, a first flexible joint connected to the first end portion, a second flexible joint connected to the second end portion, a center portion connecting the first and second flexible joint, and a first and second flexure of variable stiffnesses along their length housed within the center portion and either the first end portion or the second end portion. Next, the sliding flexure bar is inserted within a chest cavity of a patient, wherein the first end portion and the second end portion engage opposing ribs, and the center portion engages a sternum of the patient. Next, the center portion applies force against the sternum in an outward direction as the first and second flexible joint moves from a deflected position to an undeflected position. Lastly, as the center portion moves from a deflected position to an undeflected position, the first flexure and the second flexure are actuated in a manner that stiffens the first and second flexible joint.

Additional features and advantages of the present disclosure will become apparent to those skilled in the art upon consideration of the following detailed description of the illustrative embodiments exemplifying the disclosure as presently perceived.

### BRIEF DESCRIPTION OF THE DRAWINGS

The detailed description of the drawings particularly refers to the accompanying figures in which:

FIG. 1 is a perspective view of an illustrative sliding flexure bar of the present disclosure;

FIG. 2 is a cross-sectional view of one end of the sliding flexure bar of FIG. 1, the flexure slidably received within opposing hollow channels;

FIG. 3A is a perspective view of an illustrative embodiment sliding flexure;

FIG. 3B is a perspective view of another illustrative embodiment sliding flexure;

FIG. 4 is a cross-sectional view of the joint and hollow channel of the sliding flexure bar of FIG. 1, with the sliding flexure removed for clarity;

FIG. 5 is a diagrammatic side view of the sliding flexure bar of FIG. 1, illustrating the sliding flexure bar in a fully deflected state;

FIG. 6 is detail view of the sliding flexure in the joint of FIG. 5, with the sliding flexure bar shown in a fully deflected state;

FIG. 7 is a diagrammatic side view of the sliding flexure bar of FIG. 1, illustrating the sliding flexure bar in between a fully deflected state and an undeflected state;

FIG. 8 is detail view of the sliding flexure in the joint of FIG. 7, with the sliding flexure bar shown between a fully deflected state and an undeflected state;

FIG. 9 is a diagrammatic side view of the sliding flexure bar of FIG. 1, illustrating the sliding flexure bar in an undeflected state;

FIG. 10 is a detail view of the sliding flexure in the joint of FIG. 10, with the sliding flexure bar shown in an undeflected state;

FIG. 11 is a cross-sectional view of an illustrative embodiment system for actuating the sliding flexure using a spring;

FIG. 12 is a cross-sectional view of another illustrative embodiment system for actuating the sliding flexure;

FIG. 13 is a detail view of an illustrative ratcheting device of FIG. 12, configured to actuate the sliding flexure;

FIG. 14 is a cross-sectional view of another illustrative embodiment system for actuating the sliding flexure;

FIG. 15 is a detail view of another illustrative ratcheting device of FIG. 14, configured to actuate the sliding flexure;

FIG. 16 is a cross-sectional view of another illustrative embodiment system for actuating the sliding flexure using linear magnets;

FIG. 17 is a cross-sectional view of another illustrative embodiment system for actuating the sliding flexure using rotary magnets;

FIG. 18 is a cross-sectional view of another illustrative embodiment system for actuating the sliding flexure through the use of a fluid bladder;

FIG. 19 is a cross-sectional view of a further illustrative embodiment system for actuating the sliding flexure using a thermally expanding material;

FIG. 20 is a detail cross-sectional view of another illustrative embodiment system for actuating the sliding flexure using a rack and pinion; and

FIG. 21 is a detail cross-sectional view of another illustrative embodiment system for actuating the sliding flexure using a needlescopic procedure.

### DETAILED DESCRIPTION OF THE DRAWINGS

For the purposes of promoting an understanding of the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

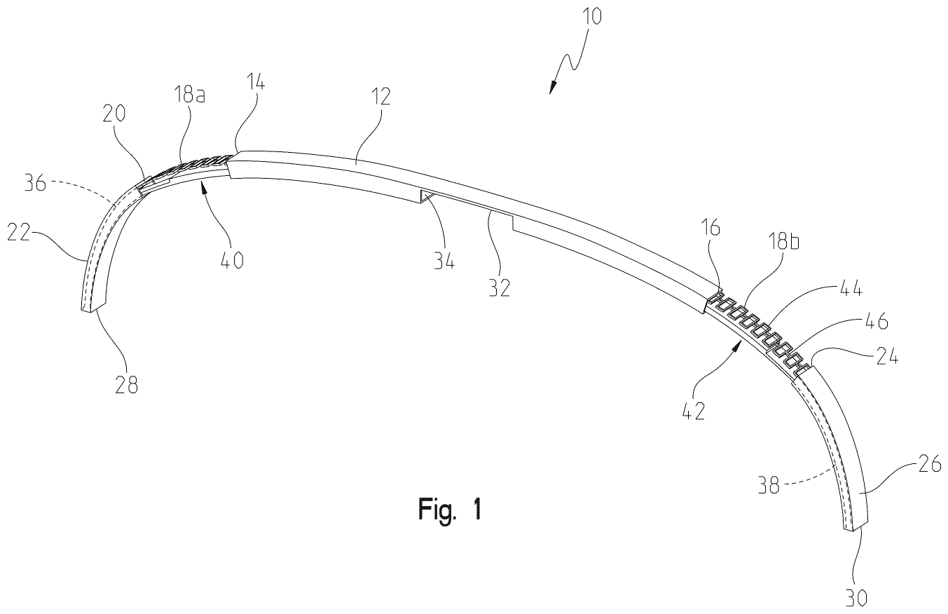

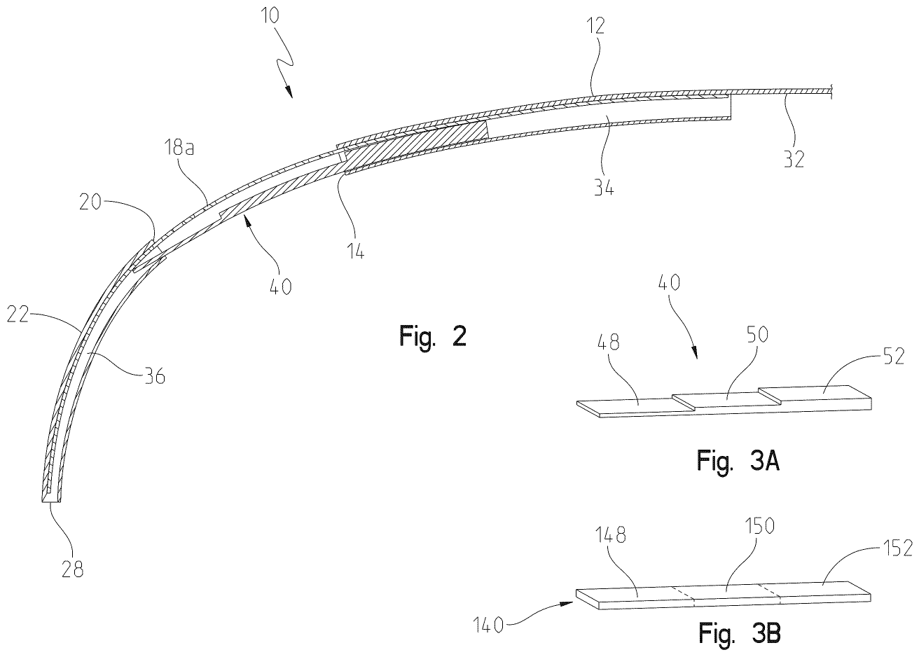

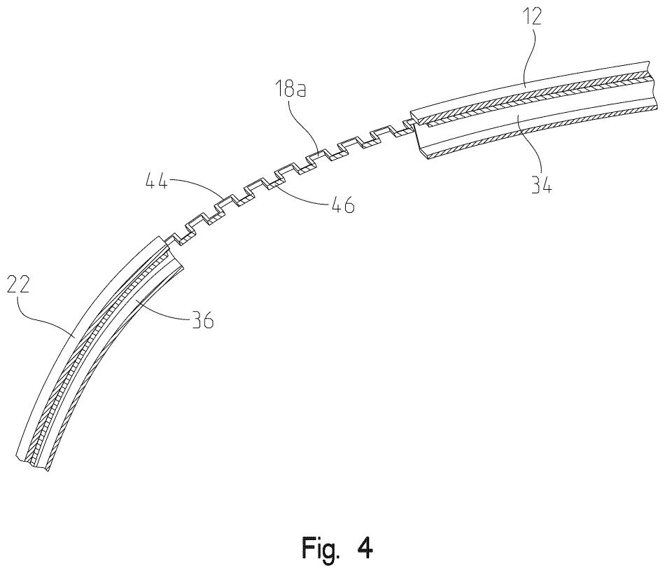

Referring initially to FIGS. 1-4, an illustrative sliding flexure bar 10 of the present disclosure is shown and configured to correct a pectus excavatum deformity in a human patient and do so in a gradual, iterative process. The illustrative bar 10 includes a center portion 12 flanked on opposing sides by joints 18a and 18b. More particularly, the center portion 12 terminates at a first end 14 and a second end 16 connecting to the joints 18a and 18b, respectively. The joints 18a and 18b extend and terminate into the distal end 20 of the first end portion 22 and to the distal end 24 of the second end portion 26, respectively. The bar 10 extends between the proximal end 28 of the first end portion 22 and the proximal end 30 of the second end portion 26. The end portions 22 and 26 are configured to perform a stabilization function, while the center portion 12 is configured to perform a correction function. In the illustrative embodiment, there may be a center plate 32, illustrated as a cut-out or gap, spanning a centered section of the center portion 12.

Still referring to FIGS. 1-4, the center portion 12 and first and second end portions 22 and 26 are hollow, thereby forming a center channel or passageway 34 in the center portion 12, a first end channel or passageway 36 in the first end portion 22, and a second end channel or passageway 38 in the second end portion 26 (FIG. 1). The center channel 34 and first end channel 36 are configured to receive a first flexure 40, and the center channel 34 and the second end channel 38 are configured to receive a second flexure 42. While received in the respective channels 36 and 38, the flexures 40 and 42 freely span the length of the joints 18a and 18b. Additionally, the flexures 40 and 42 and may slidably move within the center channel 34 and/or the respective first or second end channels 36 and 38. The flexures 40, 42 may be initially substantially received in the center channel 34 and slidably moved into the respective first and second end channels 36, 38. Alternatively, the flexures 40, 42 may be initially substantially received in the first and second end channels 36, 38 and slidably moved into the center channel 34.

The illustrative sliding flexure bar 10 retains overall dimensions similar to a conventional Nuss bar, including the overall width, overall height, and overall length. However, the conventional Nuss bar has a constant cross-section and corresponding uniform stiffness, while the illustrative sliding flexure bar 10 includes the hollow center channel 34, the first end channel 36, and the second end channel 38 cooperating with the sliding flexures 40, 42 in addition to the respective joints 18a and 18b.

With further reference to FIGS. 1-4, the joints 18a and 18b may each be a torsional joint assembly, hereafter referred to as a Lamina Emergent Torsional (LET) joint. A LET joint is formed by having at least two parallel hinge segments 44 connected to each other by a substantially orthogonally placed connecting member 46. The hinge segments 44 may be in the shape of a rectangle as illustrated, but may take on the form of another shape depending on the desired characteristics of the joint 18. In the illustrative embodiment, a LET joint allows for higher flexibility of the joint 18 while also reducing stress across the length of the joint 18. It may be appreciated that the joints 18 could be replaced with other compliant mechanisms known to one skilled in the art including, but not limited to, pin joints or a thin compliant plate. While the illustrative embodiment shows that the LET joint is affixed to a particular face of the center portion 12 and first and second end portions 22 and 26, the joint 18 may be affixed to any face.

The center portion 12 and the end portions 22 and 26 of the illustrative bar 10 have a stiffness greater than that of the joints 18, allowing the joints 18 to flex and bend while the center portion 12 and end portions 22 and 26 remain stable and supportive. A compliant center plate 32 may also be centrally formed on the center portion 12 that may nominally flex to withstand a bending load placed on the center portion 12.

The illustrative bar 10 may be formed by a single sheet of material. Alternatively, the components may be manufactured and independently assembled through welding or similar manufacturing method known to those skilled in the art. It can be recognized that the bar 10 and the flexures 40, 42 may be formed of a biocompatible material, such as Ti-6Al-4V (Ti-64), or any such biocompatible material with similar stiffness and yield strength properties. Further, the bar 10 and the flexures 40, 42 may be covered in a biocompatible silicon sheath, or a material of similar properties that could protect the bar 10 from tissue ingrowth.

FIG. 3A is an illustrative embodiment of the flexure 40. The illustrative flexure 40 is a single piece of material featuring machined areas of different thicknesses. The first or proximal end zone 48 is the thinnest area and allows for the largest amount of flexibility. The second or center zone 50 is of intermediate thickness and is stiffer than the first zone 48. The third or distal end zone 52 has the greatest thickness and is the stiffest area of the flexure 40.

In an alternative embodiment, shown in FIG. 3B, the flexure 140 may be one uniform thickness with a first or proximal end zone 148, a second or center zone 150, and a third or distal end zone 152, each made from a material of a different flexibility. The first zone 148 being the least stiff and most compliant, whereas the third zone 152 is the stiffest and least compliant. It can be appreciated that although the illustrated embodiments show three zones of variable stiffness, there could be as few as two zones or greater than three zones. While illustrated in FIGS. 3A and 3B that the first or proximal end zones 48, 148 have the least stiffness (i.e., the most compliant) and the third or distal end zones 52, 152 have the greatest stiffness, it can be appreciated that the flexures 40, 42 may have an opposite configuration in which the first or proximal end zones 48, 148 are of the greatest stiffness and the third or distal end zones 52, 152 have the least stiffness (i.e., the most compliant).

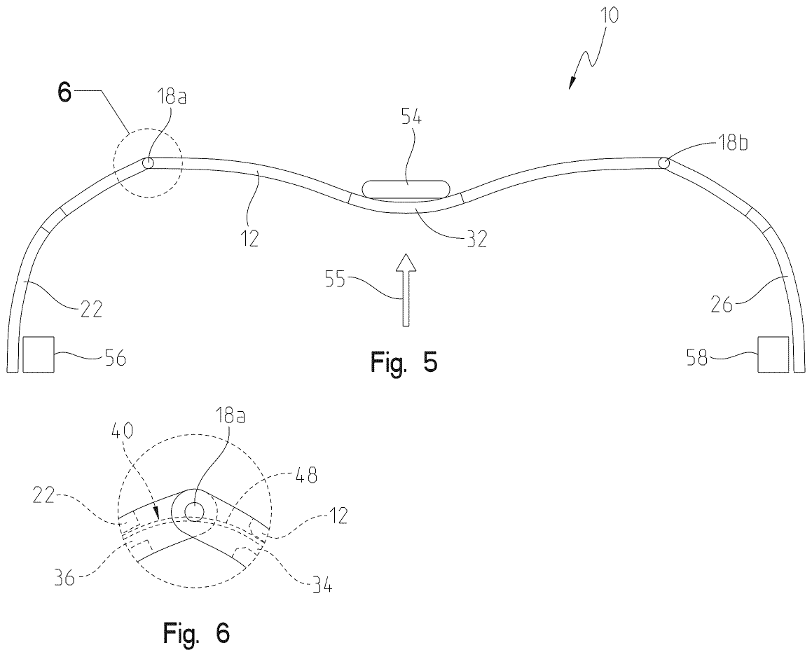

FIG. 5 is a diagrammatic representation of the illustrative sliding flexure bar 10 in a deflected state when placed under a load from a patient's sternum 54. The bar 10 is placed into a patient's rib cage such that the center plate 32 is beneath and supporting the sternum 54 while applying an outward force (as shown by arrow 55). The first end portion 22 and the second end portion 26 are supported by a first rib 56 and a second rib 58, respectively. FIG. 6 shows a detail view of the illustrative joint 18a in the deflected state of FIG. 5. The first zone 48 of the illustrative flexure 40 spans the distance of the joint 18, providing the most flexibility and compliance.

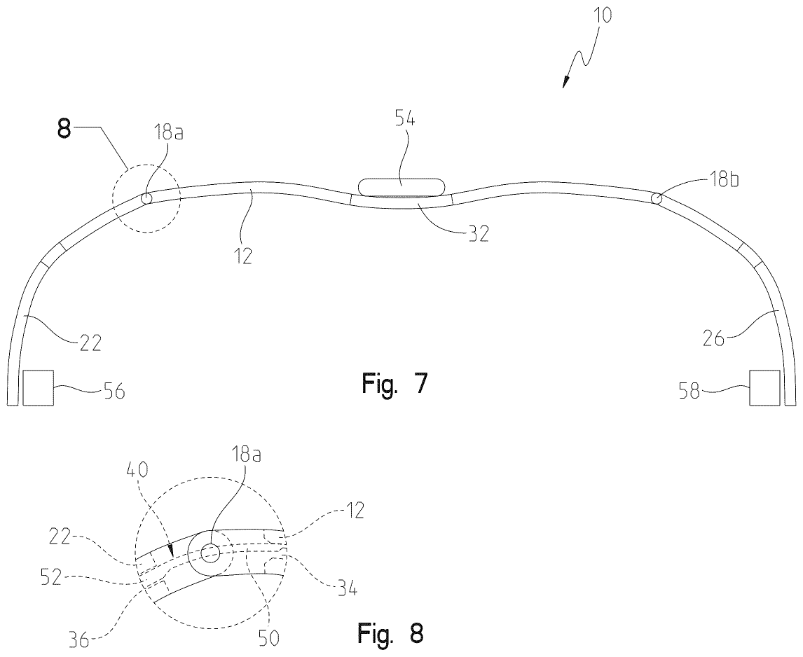

FIG. 7 is a diagrammatic representation of the illustrative sliding flexure bar 10 in between a deflected state and an undeflected state when placed under a load from the patient's sternum 54. FIG. 8 shows a detail view of the illustrative joint 18a in the intermediate state of FIG. 7. The second zone 50 of flexure 40 has been actuated and slidably moved within the center channel 34 and the first end channel 36, and spans the distance of the joint 18a, providing increased stiffness and thus applying more force on the sternum 54.

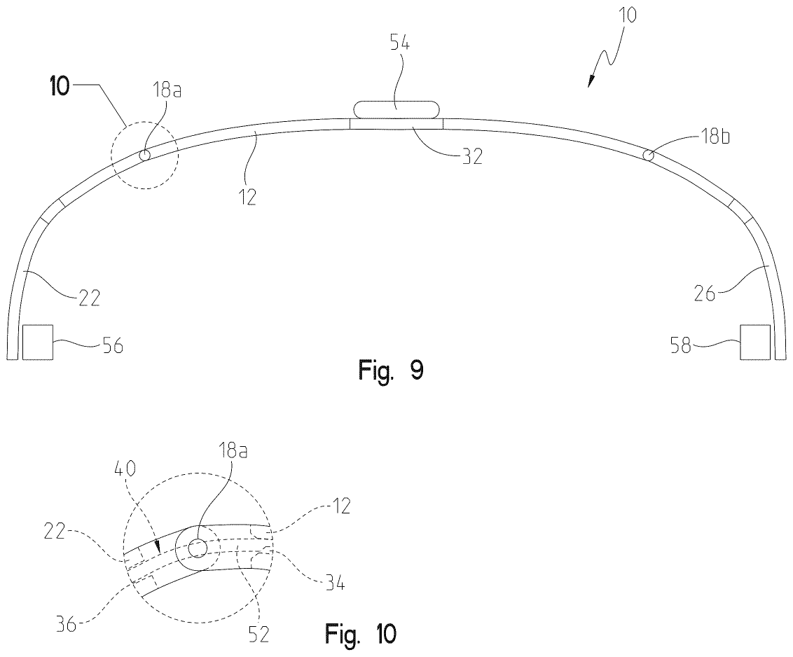

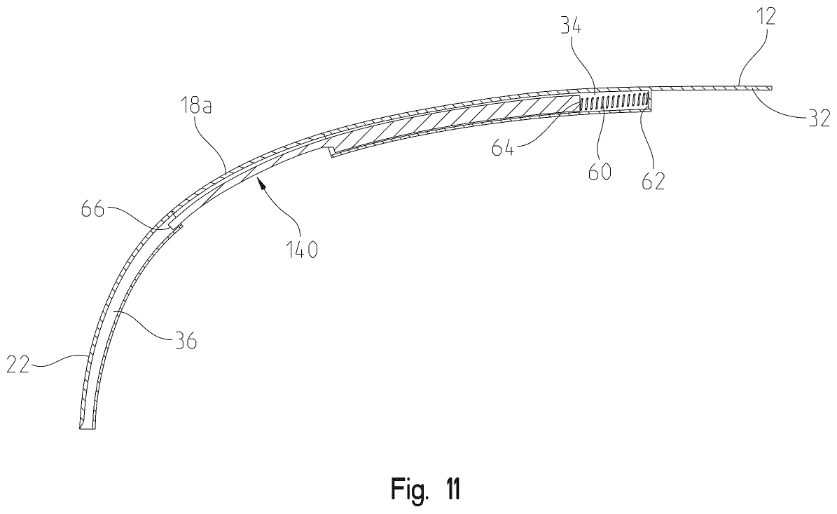

FIG. 9 is a diagrammatic representation of the illustrative sliding flexure bar 10 in an undeflected state after applying a load to the sternum 54 and correcting the chest wall deformity. FIG. 10 shows a detail view of the illustrative joint 18 in the undeflected state of FIG. 9. The third zone 52 of flexure 40 has been actuated and slidably moved within the center channel 34 and the first end channel 36 and spans the distance of the joint 18, providing the greatest stiffness. This prevents any chest wall movement backwards, providing a permanent correction of the chest wall deformity.

FIG. 11 shows an illustrative embodiment of an actuation system and related method to slide the flexure 140 within the bar 10. Illustratively, a spring includes a first end coupled to the channel wall 62, on one side and a second end coupled to the first flexure end 64. The spring 60 may be made from nitinol or any other alloy with characteristics of shape memory. The spring 60 may start in a compressed state at body temperature and upon heating may expand, thus sliding the flexure 140 from the center channel 34 into the respective first end channel 36 or second end channel 38. Alternatively, the spring 60 could be received in the respective first end channel 36 and second end channel 38 and attached to the second flexure end 66. The spring 60 may start in an elongated state and upon external healing begin to compress, thus pulling the flexure 140 from the center channel 34 into the first end channel 36 or second end channel 38.

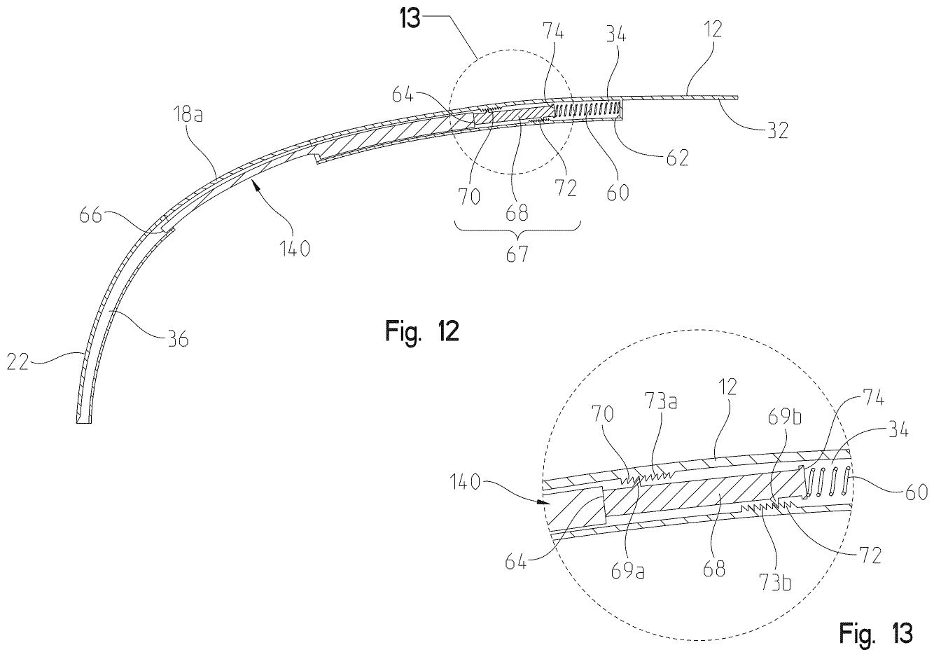

FIGS. 12 and 13 show an illustrative embodiment of a shape memory alloy actuation system and related method of the flexure 140 featuring a locking rachet device 67. The ratchet device 67 includes a pawl member 68 supporting pawls 69a and 69b, a top ratchet 70, and a bottom ratchet 72. In the illustrative embodiment, the rear end 74 of the ratchet device 67 is coupled to the spring 60. As the spring 60 advances the pawl member 68, pawl 69a advances over a tooth 73a of the top ratchet 70 and pawl 69b advances over a tooth 73b of the bottom ratchet 72. This movement advances the pawl member 68 which in turn abuts the first flexure end 64 and advances the flexure 140. There may be a plurality of teeth 73 on each ratchet 70 and 72 and at least two. In this manner, any movement of the flexure 140 back into the center channel is prevented.

It can be appreciated that the ratchet device 67 and spring 60 assembly may be housed in the first end channel 36 or second end channel 38 to retract the flexure 140 from the center channel 34 into the first end channel 36 or second end channel 38. As the flexure 140 is retracted by the second flexure end 66 being coupled to the pawl member 68, pawl 69a advances over a tooth of the forward ratchet 70 and pawl 69b advances over a tooth of the rear ratchet 72. Again, any movement of the flexure 140 back into the center channel is prevented.

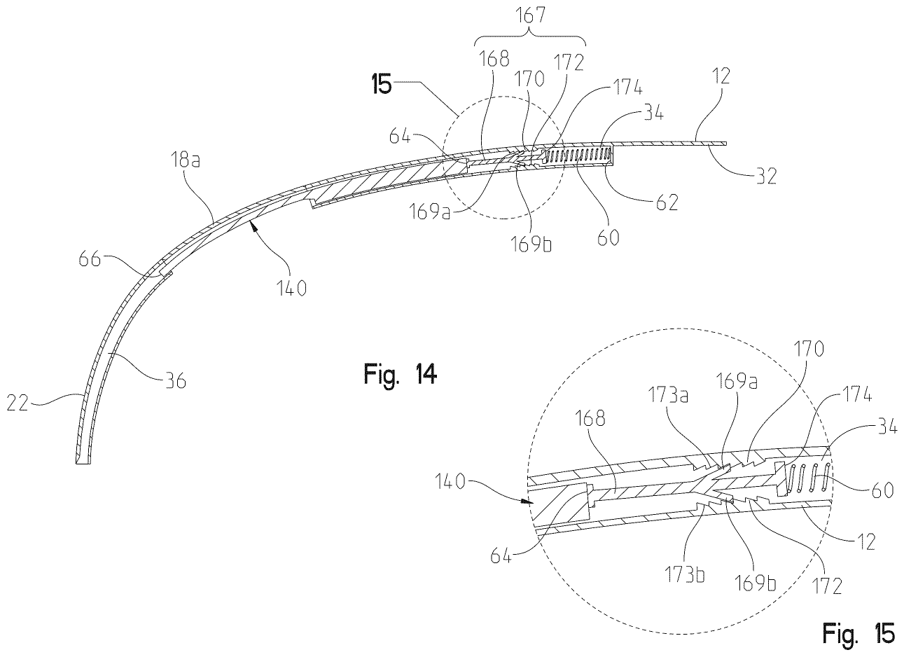

FIGS. 14 and 15 show an alternative embodiment for a ratchet device 167 to be used with a shape memory alloy actuation method. In this embodiment, the ratchet device 167 includes a pawl member 168 supporting pawls 169a and 169b, a top ratchet 170, and a bottom ratchet 172. The rear end 174 of the ratchet device 167 is attached to the spring 60 and the pawl member 168 abuts the first flexure end 64.

As the spring 60 expands, the pawl member 168 advances the flexure 140 out of the center channel 34. As the spring 60 advances the pawl member 168, pawl 169a advances over a tooth 173a of the top ratchet 170 and pawl 169b advances over a tooth 173b of the bottom ratchet 172. The advancement of the pawls 169 over the teeth 173 occurs simultaneously. This movement advances the pawl member 168 which in turn abuts the first flexure end 64 and advances the flexure 140. There may be a plurality of teeth 173 on each ratchet 170 and 172 and at least two. In this manner, any movement of the flexure 140 back into the center channel is prevented.

It can be appreciated that the ratchet device 167 and spring 60 assembly may be housed in the first end channel 36 or second end channel 38 to retract the flexure 140 from the center channel 34 into the first end channel 36 or second end channel 38. As the flexure 140 is retracted by the second flexure end 66 being coupled to the pawl member 168, pawl 169a advances over a tooth of the forward ratchet 170 and pawl 169b advances over a tooth of the rear ratchet 172. Again, any movement of the flexure 140 back into the center channel is prevented.

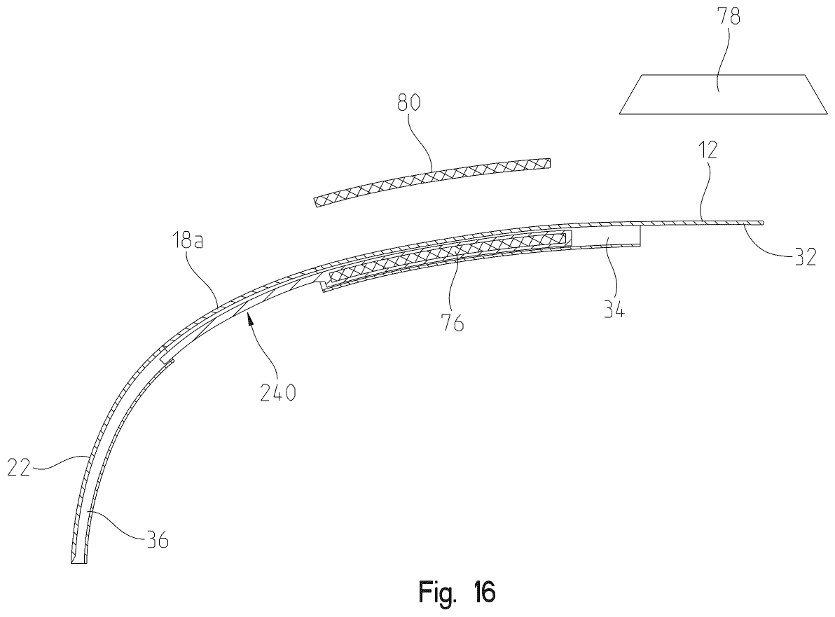

FIG. 16 shows an illustrative embodiment for actuating the flexure 240 in which a magnet 76 is coupled to the flexure 240. In this embodiment, a pulling device 78 may be employed to pull the chest wall forward. Illustratively, the pulling device may be a suction device, such as a bell vacuum or any like device known to those who are skilled in the art. Next, an external magnet 80 may be used to slidably advance the flexure to the desired stiffness zone. This may be repeated until the position of the chest wall has been permanently corrected.

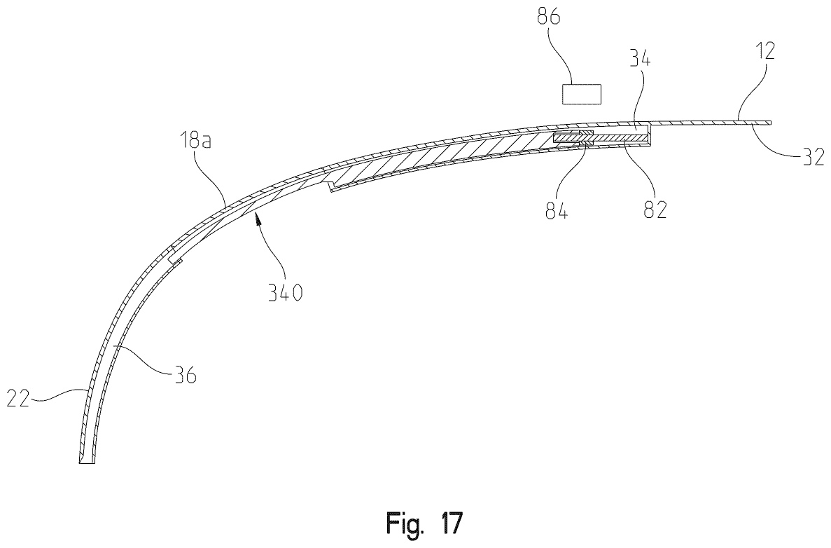

FIG. 17 shows an embodiment for actuating the flexure 340 by using rotary magnets. Illustratively, a guide rod 82 is housed within the center channel 34 and continues into a cavity in the flexure 340. Alternatively, the guide rod 82 could be housed in the first and second end channels 36, 38. A magnetic collar 84 is located on the guide rod 82. An external magnetic device 86 may be used to create alternating magnetic fields, this impact of which would turn the magnetic collar 84, advancing the magnetic collar 84 on the guide rod, and thus advancing the flexure 340.

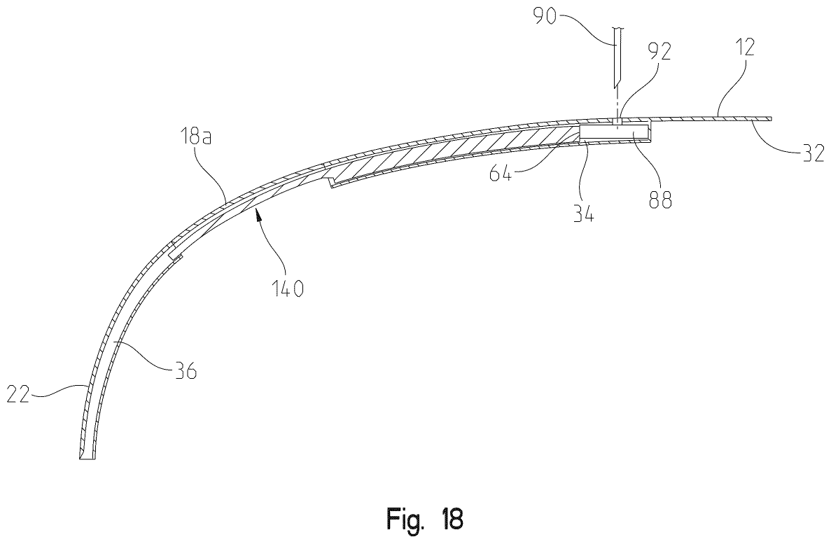

FIG. 18 shows an embodiment for actuating the flexure 140 by utilizing a fluid bladder 88. The bladder 88 is housed in the center channel 34 and abuts the first flexure end 64. A needle 90 may be advanced through a self-sealing valve 92 and fill the bladder 88 with a fluid, expanding the bladder 88 and advancing the flexure 140 into the first and second end channels 36, 38. The fluid may be saline, or any similar biocompatible fluid known to those skilled in the art. It can be appreciated that the bladder 88 may be housed in the first or second end channel 36, 38 and advance the flexure 140 into the center channel 34.

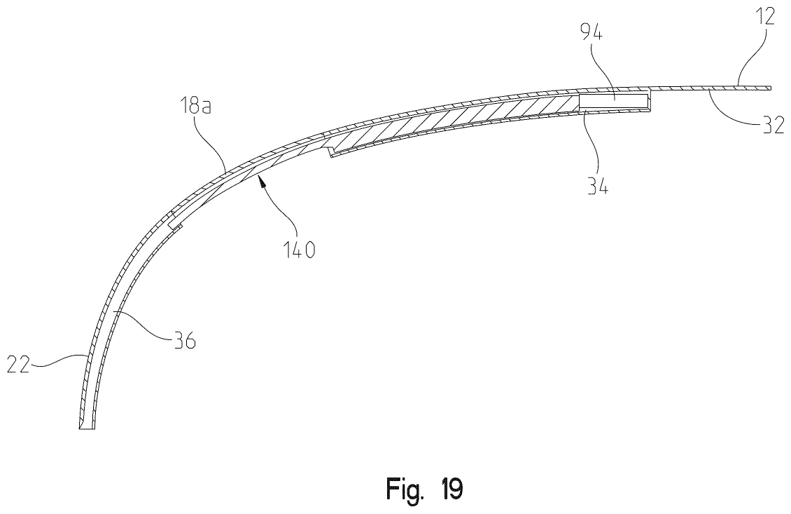

FIG. 19 shows an illustrative embodiment for actuating the flexure 140 by utilizing a thermally actuated material 94 (e.g., a polymer housed in the center channel 34). Once heated externally, the thermally actuated material 94 will expand, advancing the flexure 140. A ratcheting mechanism as described above could be employed to lock the flexure in place after the thermally actuated material 94 has cooled and shrunk to its original size. It can be appreciated that the thermally actuated material 94 may be housed in the first and second end channels 36, 38 and advance the flexure 140 into the center channel 34.

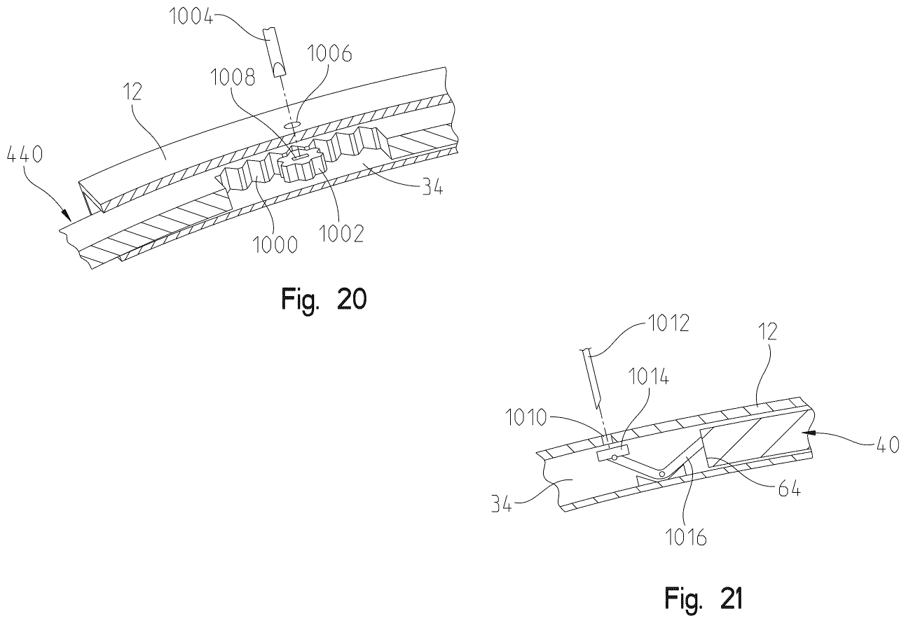

FIG. 20 shows an illustrative embodiment for actuating the flexure 440 by rack 1000 and pinion 1002. The flexure 440 features a rack 1000 in the portion of the flexure 440 that is housed in the center channel 34. Coupled to the walls of that channel 34 is the pinion 1002 that is engaged with the rack 1000. To actuate the flexure 440, a screwdriver 1004 is utilized. On the center portion 12 is a port 1006 that accepts the screwdriver 1004. Once the port 1006 accepts the screwdriver 1004, there is a groove 1008 on the pinion 1002 in which the screwdriver 1004 may engage and rotate, advancing the flexure 440. It can be appreciated that the rack 1000 and pinion 1002 may be housed in the first and second end portions 36, 38 and thereby advancing the flexure 440 into the center channel 34 upon a rotation of the pinion 1002.

FIG. 21 shows an illustrative embodiment for actuating the flexure 40 through a needlescopic procedure. A port 1010 may be located on the center portion 12 that accepts a needle 1012. Once inserted through the port 1010, the needle 1012 may actuate a button 1014. By actuating the button 1014, a mechanism 1016 is triggered. The mechanism 1016 abuts the first flexure end 64 and, once triggered, with advance the flexure 40. The mechanism 1016, while not limited to the following examples, could be a lever, a lock release, a spring release, a rachet system, or any like mechanisms known to those skilled in the art.

An illustrative method for correcting pectus excavatum may include a surgical procedure in which the bar 10 of the present disclosure is weaved into a patient's ribcage such that the center portion 12 is placed posteriorly to the sternum 54. First and second end portions 22 and 26 rest on support ribs 56 and 58. When implanted, the center portion 12 of the bar 10 deflects under the load of the sternum 54. The load created by the sternum 54 is applied to the support ribs 56 and 58 whereas the deflected bar 10 applies an opposite force on the sternum 54 as it returns to an undeflected state. In the deflected state, the forces applied resemble three-point bending.

In the deflected state, joints 18 and the center cut 32 may bend and decrease the stress put on the bar 10. The third zone 52 of flexures 40, 42 may be housed in the first and second end channels 36, 38 such that the first zone 48 of the bar 10 spans the length of the joint 18, allowing for the largest amount of deflection. Alternatively, the third zone 52 of the flexures 40, 42 may be housed in the center channel 34.

Once the bar 10 is placed, the force applied on the sternum 54 decreases the stiffness of the chest wall, subsequently correcting the position of the sternum 54 as the bar 10 relaxes into its undeflected state. During this relaxation, the flexures 40, 42 may be actuated by any of the previously discussed methods or those known to those who are skilled in the art. In actuating the flexures 40, 42, the second zone 50 and third zone 52 of the flexure are slidably moved into the joint 18. In each successive zone, the joint 18 becomes stiffer moving the bar 10 from the deflected state to an undeflected state. As the flexures 40, 42 are actuated, a ratchet device may lock the flexures 40, 42 in place to prevent a regressive movement and to steadily apply force on the sternum 54. Finally, the actuation methods may employ a pulling device 78 to bring the chest wall forward and advance the flexures 40, 42. In reaching the undeflected state, the bar 10 corrects the position of the chest wall, thus correcting the deformity.

Although the invention has been described in detail with reference to certain preferred embodiments, variations and modifications exist within the scope of the invention as described and defined in the following claims.

What is claimed is:

1. A sliding flexure bar for pectus excavatum repair, the sliding flexure bar comprising:
a first end portion extending between a proximal end and a distal end, the first end portion including a channel;
a second end portion in spaced relation to the first end portion, the second end portion extending between a proximal end and a distal end, and including a channel;
a first flexible joint extending from the distal end of the first end portion;
a second flexible joint extending from the distal end of the second end portion;
a center portion connecting to the first flexible joint at a first end and the second flexible joint at a second end, the center portion including a channel, the flexible joints permitting the center portion to be moveable between a undeflected state and a deflected state, the center portion extending outwardly in a convex manner from the first and second flexible joint in the undeflected state, and the center portion extending inwardly in a concave manner from the first and second flexible joint in the deflected state;
a first flexure received in the channel of the center portion on a first end and received in the channel of the first end portion on a second end, the first flexure having variable stiffness along its length; and
a second flexure received in the channel of the center portion on a first end and received in the channel of the second end portion on a second end, the second flexure having variable stiffness along its length.

2. The sliding flexure of claim 1, wherein the first flexure the second flexure are slidably actuated from a zone of low stiffness to a zone of higher stiffness, stiffening the first and second flexible joints to move the center portion into an undeflected state.

3. The sliding flexure bar of claim 1, wherein in the deflected state of the center portion, the first and second flexure are configured to be in a reduced stiffness configuration;

4. The sliding flexure bar of claim 1, wherein the bar is integrally formed of a biocompatible material.

5. The sliding flexure bar of claim 1, wherein the first end portion and the second end portion are stabilization members, and the center portion is a correction member.

6. The sliding flexure bar of claim 5, wherein the first end portion, the second end portion, and the center portion each have a stiffness greater than the first and second flexible joints.

7. The sliding flexure bar of claim 1, wherein the flexible joints are lamina emergent torsional joints.

8. The sliding flexure bar of claim 1, wherein a ratchet device is removably coupled to the first and second flexures.

9. The sliding flexure bar of claim 8, wherein the first and second flexures are actuated by a shape memory alloy.

10. The sliding flexure bar of claim 8, wherein the first and second flexures are actuated by a thermally actuated material.

11. The sliding flexure bar of claim 1, wherein the first and second flexures support magnets, wherein the use of an external magnet may actuate the first and second flexures.

12. A system for pectus excavatum repair, the system comprising:
a sliding flexure bar including:
a first end portion extending between a proximal end and a distal end;
a second end portion in spaced relation to the first end portion, the second end portion extending between a proximal end and a distal end;
a first flexible joint extending from the distal end of the first end portion;
a second flexible joint extending from the distal end of the second end portion;
a center portion connecting to the first flexible joint at a first end and the second flexible joint at a second end, the flexible joints permitting the center portion to be moveable from a undeflected state to a deflected state, and vice versa, the center portion extending outwardly in a convex manner from the first and second flexible joint, and the center portion extending inwardly in a concave manner from the first and second flexible joint in the deflected state;
a first flexure including a first end received in the center portion, and a second end received in the channel of the first end portion, the first flexure having variable stiffness along its length;
a second flexure including a first end received in the center portion, and a second end received in the second end portion, the second flexure having variable stiffness along its length; and
a pulling device;
wherein the pulling device is configured to be placed externally of the sliding flexure bar and upon actuation increases the force that the center portion exerts to move from the deflected state to the undeflected state; and
wherein increasing the force that the center portion exerts assists actuating the first and second flexures, moving the center portion from a deflected state to an undeflected state.

13. The system of claim 12, wherein the pulling device comprises a vacuum bell for pulling a vacuum.

14. The system of claim 12, wherein the sliding flexure bar is integrally formed of a biocompatible material.

15. The system of claim 12, wherein the first end portion and the second end portion are stabilization members, and the center portion is a correction member.

16. The system of claim 15, wherein the first end portion, the second end portion, and the center portion each have a stiffness greater than the first and second flexible joints.

17. The system of claim 12, wherein the first flexure supports a first magnet, and the second flexure supports a second magnet.

18. The system of claim 17, further comprising an external magnet, wherein after the pulling device increases the force that the center portion exerts the external magnet may be used to magnetically actuate the first and second flexures supporting the first and second magnets.

19. The system of claim 12, wherein the first and second flexures are further actuated by a shape memory alloy.

20. A method of correcting pectus excavatum, the method comprising the steps of:
providing a sliding flexure bar including a first end portion, a second end portion in spaced relation to the first end portion, a first flexible joint connected to the first end portion, a second flexible joint connected to the second end portion, a center portion connecting the first and second flexible joint, and a first and second flexure of variable stiffnesses along their length housed within the center portion and either the first end portion or the second end portion;
inserting the sliding flexure bar within a chest cavity of a patient, wherein the first end portion and the second end portion engage opposing ribs, and the center portion engages a sternum of the patient;
wherein the center portion applies force against the sternum in an outward direction as the first and second flexible joint moves from a deflected position to an undeflected position; and
wherein as the center portion moves from a deflected position to an undeflected position, the first flexure and the second flexure are actuated in a manner that stiffens the first and second flexible joint.

21. The method of claim 20, wherein the first end portion, the second end portion, and the center portion each have a stiffness greater than the first and second flexible joints.
